(19) United States
(12) Patent Application Publication
(10) Pub. No.: US 2002/0058317 A1
(43) Pub. Date: May 16, 2002

(54) Process for the fusion of dendritic cells with abnormal tissue cells, in particular tumor cells and media for such a process

(76) Inventor: Ulrich Zimmermann, Waldbrunn (DE)

Correspondence Address: RANKIN, HILL, PORTER & CLARK, LLP, 700 HUNTINGTON BUILDING, 925 EUCLID AVENUE, CLEVELAND, OH, 44115-1405, US

(21) Appl. No.: 09/951,207

(22) Filed: Sep. 13, 2001

(30) Foreign Application Priority Data
Sep. 14, 2000 (DE) 10047272.9

(51) Int. Cl. C12N 5/00

(52) U.S. Cl. 435/173.1; 435/346; 514/23

(57) ABSTRACT

A process for electrofusion of dendritic cells with abnormal cells, particularly tumor cells, that achieves a higher yield of fusion product, as well as a media that can be used in the process. The dendritic cells are combined with tumor cells and washed, if necessary, with a washing medium and then centrifuged. The resulting pellet is suspended in an electrofusion medium and then treated by electrofusion. The electrofusion medium contains sugar (20 mM/L to 150 mM/L), magnesium salt (0.05 mM/L to 1 mM/L) and calcium salt (0.01 mM/L to 1 mM/L), in pyrogen-free, sterile distilled water. The washing medium is a solution containing sugar (270 mM/L to 310 mM/L), magnesium salt (0.05 mM/L to 1 mM/L) and calcium salt (0.01 mM/L to 1 mM/L), in pyrogen-free, sterile distilled water.

The invention relates to a process according to the superordinate concept of claim 1.

Such generic procedures provide for the production of fused cells (the fusion product) comprised of dendritic cells and in particular, tumor cells. Dendritic cells are immunoactive cells that can present antigens on their surfaces and, depending on the antigen presented, exhibit corresponding immunostimulatory properties.

Fusion products comprised of dendritic cells and tumor cells can be used in the treatment of tumors. In this context, reference is made to the article by Kugler et al. in “Nature Medicine, Vol. 6. N^{o} 3, pages 332 to 336 (2000) which describes the use of dendritic cells fused with tumor cells in the treatment of metastasizing renal cell carcinoma.

In the known process tumor cells are taken from a patient and fused with dendritic cells by electrofusion techniques.

The dendritic cells can be the patient's own or can originate from other human beings or from animals.

However, the dendritic cells as well as the tumor cells must be irradiated, for example, in order to kill viruses.

Electrofusion relates to a known, generally multi-step process. A more complete description of the process can be found, for example, in the book by U. Zimmerman & G. Neil, “Electromanipulation of Cells,” CRC-Pub, Bocaration [sic], Florida, 1996.

In an initial step (pre-alignment) the cells are brought into close contact with each other by using dielectrophoresis. In a second step (fusion pulse) a pulse is applied, resulting in a permeation of the cell membrane, whereupon the cell membranes and thus the cells can then fuse. In a third step (post-alignment), in order to assure stabilization of the fusion partners, the fused cells are maintained in contact with each other using dielectrophoresis until the fusion process is completed.

After irradiation, the fusion products formed in this fashion (comprised of tumor cells and dendritic cells) are injected into the patient and trigger an immunostimulatory effect in the body.

The disadvantage in the process described above is the fact that the conditions selected for production of the fusion product do not provide optimal results.

The object of the invention is to provide a process for electrofusion of dendritic cells with abnormal cells, in particular with tumor cells, that achieves a higher yield of fusion product. A further object of the invention is to provide media that can be used in said process.

Said object is achieved using a process characterized by the disclosures of claim 1 and using media according to claims 17 and 18.

In the inventive process, as in the prior art, the dendritic cells are mixed with the cells from abnormal tissue, in particular tumor cells.

Preferably the mixture ratio relative to cell numbers is 1 to 1. Particularly preferred is the use of a cell count of 2×10⁶ per line (thus, a total of 4×10⁶ cells in the cell mixture).

The cell mixture is then, if required, washed with an isoosmolar wash medium, centrifuged and then re-suspended. The re-suspended cell mixture is then placed in an electrode chamber, for example, and there exposed to electrical conditions that result in the fusion of the cells with each other.

According to the invention, the electrofusion medium is a solution comprised of the following components in pyrogen-free, sterile distilled water:

20 mM/L-150 mM/L sugar,

0.05 mM/L-1 mM/L magnesium salt and

0.01 mM/L-1 mML calcium salt.

With respect to medical application, the electrofusion medium is manufactured preferably by mixing of infusion solutions of appropriate components.

Preferably, sorbitol is used as the sugar in a concentration of 75 mM/L added to an infusion solution that is available from the Serum-Werk Bemburg AG under the name “Sorbitol-Infusion Solution 40”. In lieu of said solution, other solutions that contain other sugars, for example glucose can also be used.

The magnesium salt to be used is preferably magnesium-L-hydrogen gluconate solution which, for example, is available from the Verla-Pharm GmbH company under the name of “Magnesium Veria.” Preferably, a concentration of 0.5 mM/L magnesium salt is used. It is also conceivable, of course, that alternative solutions containing alternative magnesium salts, for example magnesium chloride be used.

The calcium salt is preferably added as a calcium gluconate disaccharide solution in a preferred concentration of 0.1 mM/L. A suitable infusion solution is “Calcium Braun 10%” available from the Braun company of Melsungen. Here, too, alternative solutions containing alternative calcium salts in alternative concentrations can be used.

If the aforesaid conditions are selected as optimal conditions, then an electrofusion medium having a conductivity of approximately 85 μS, a pH of approximately 6.6 and an osmolarity of approximately 70 mOsm is achieved.

The osmolarity of the electrofusion medium can be varied in the range of approximately 20 mOsm-150 mOsm within the inventive scope by way of the sugar concentration depending on the cells to be fused.

A further embodiment of the invention relates to the washing medium used during the process, said washing medium having isoosmolar properties and is preferably a solution comprised of the following components in pyrogen-free, sterile distilled water:

270 mM/L-310 mM/L sugar,

0.05 mM/L-1 mM/L magnesium salt and

0.01 mM/L-1 mML calcium salt.

With respect to medical application, the washing medium is manufactured preferably by mixing of, for example, the aforesaid infusion solutions. Here, too, alternative solutions having alternative compositions can be used.

If a sugar concentration of 290 mM/L is used, then a washing medium having a conductivity of approximately 75 μS, a pH of approximately 6.3 and an osmolarity of approximately 280 mOsm is achieved.

The combination of the indicated washing solution with the inventively used electrofusion solution has been shown to be particularly advantageous.

In both media trehalose can be used in part as the sugar or the sugar can in part be substituted with polyethylene glycol.

A further enhancement of the fusion results can be achieved if, at the time of electrofusion special electrical conditions are established.

Conventionally, as disclosed in the forgoing, a 3-step electrical treatment is performed. In a first step the cells are situated adjacent to one another (pre-alignment). In a second step the cell membranes are permeated (the fusion pulse). In a third step, the permeated cells are maintained in contact until stabilization of fusion (post-alignment). In this fashion, the electrofusion can be accomplished in a conventional electrode chamber. An advantageous embodiment of the invention provides for the use of micro- or nanoelectrode structures that include deposited electrodes using semiconductor technology.

The use of micro- or nanostructures allows the process to be automated. In addition, elegant targeted paired fusions of both cell types can be achieved in this fashion. This can, of course, be achieved also by using alternative technologies such as, for example, by the use of filters, mechanical apparatuses or even by the use of ultrasound, to mention only a few example,

All voltage indications made in the following refer to an electrode spacing of 0.2 mm. Said spacing is not compulsory. Chambers or structures with alternative electrode spacing can certainly be used. In such cases, merely the indicated voltages must be recalculated.

In advantageous embodiments of the invention the pre-alignment is expected to be carried out at an alternating voltage of between 5-20 volts in a period of between 30 seconds and 3 minutes. The frequency of the voltage can be selected to be between 1 kHz and 4 MHz. The preferred optional conditions are 10 V (2 MHz) over a period of 60 seconds.

In the second step, according to the invention, at least on pulse using a voltage of between 15 and 50 volts direct current voltage and a period of 15 μsec to 1 msec is applied Preferably, a pulse using a voltage of 20 volts for a period of 40 μsec is used. The pulse is preferably a square-wave pulse. It is also conceivable, that the voltage decrease or increase during the pulse.

The post-alignment is performed at an alternating voltage of between 2.5-10 volts over a period of between 5 seconds and 60 seconds. The frequency of the voltage can be optionally selected between 1 kHz and 4 MHz. The preferred optional conditions are 5 V (2 MHz) over a period of 30 seconds.

In the following the invention is more fully explained read together with an example.

1. A process for the fusion of dendritic cells with tumor cells in which dendritic cells are combined with tumor cells, if necessary washed using a washing medium and then centrifuged, the pellet suspended in an electrofusion medium and then treated by electrofusion, wherein the electrofusion medium is a solution containing the following components in pyrogen-free, sterile distilled water:
20 mM/L-150 mM/L sugar,
0.05 mM/L-1 mM/L magnesium salt and
0.01 mML-1 mML calcium salt.

2. A process according to claim 1, wherein the sugar concentration is 75 mM/L, the magnesium salt concentration is 0.5 mM/L and the calcium salt concentration is 0.1 mM/L.

3. A process according to claim 1, wherein the sugar used is sorbitol or glucose.

4. A process according to claim 1, wherein the sugar used is partly trehalose.

5. A process according to one of the aforesaid Claims, wherein the electrofusion medium has a conductivity of 70-90 μS, a pH level of approximately 6.6-7.0 and an osmolarity of 70 mOsm.

6. A process according to one of the aforesaid Claims, wherein the washing medium is a solution comprised of the following components in pyrogen-free, sterile distilled water:
270 mM/L-310 mM/L sugar,
0.05 mM/L-1 mM/L magnesium salt and
0.01 mM/L-1 mML calcium salt

7. A process according to claim 8, wherein the washing medium has a conductivity of 75 μS, a pH level of approximately 6.3-70 and an osmolarity of 280 mOsm.

8. A process according to claim 1, wherein in the washing medium and/or the electrofusion medium the sugar is partly replaced by polyethylene glycol.

9. A process according to one of the aforesaid Claims, wherein in a first step (pre-alignment) in the electrofusion process a field intensity of from 250 to 1,000 Volts/cm alternating current voltage is applied over a period of between 30 seconds and 3 minutes.

10. A process according to one of the aforesaid Claims, wherein in a second step (fusion pulse) in the electrofusion process at least one pulse using a field intensity of 750 to 2,500 volts/cm direct current voltage is applied over a period of 15 μsec to 1 msec.

11. A process according to claim 1, wherein in a third step (post-alignment) in the electrofusion process a field intensity of between 125 and 500 volts/cm alternating current voltage is applied over a period of between 5 seconds and 60 seconds.

12. A process according to claim 1, wherein the frequency in the first and the third steps in the electrofusion process is between 1 kHz and 5 MHz.

13. A process according to claim 1, wherein the electrofusion is carried out in a micro- or nanoelectrode structure that has electrodes manufactured using semiconductor technology.

14. A process according to claim 13, wherein the electrofusion is automatedly carried out.

15. A process according to claim 1, wherein the cells of the various cell types brought together and are targetedly paired.

16. A process according to claim 1 wherein following the third step the cells are allowed to rest for 10 minutes at room temperature, thereafter physiological buffered conditions are established, and then incubated for at least 30 minutes at 37° C.

17. An electrofusion medium for use in a process according to claim 1, wherein said medium is a solution comprised of the following components in pyrogen-free, sterile distilled water.
20 mM/L-150 mM/L sugar,
0.05 mM/L-1 mM/L magnesium salt and
0.01 mM/L-1 mML calcium salt.

18. An washing medium for use in a process according to claims 1-16, wherein said medium is a solution comprised of the following components in pyrogen-free, sterile distilled water:
270 mM/L-310 mM/L sugar,
0.05 mM/L-1 mM/L magnesium salt and
0.01 mM/L-1 mML calcium salt.
